(19) United States
(12) Patent Application Publication
(10) Pub. No.: US 2008/0233206 A1
(43) Pub. Date: Sep. 25, 2008

(54) COMPOSITIONS AND METHODS FOR ANTI-INFLAMMATORY TREATMENTS

(75) Inventor: Piotr Chomczynski, Cincinnati, OH (US)

Correspondence Address: FROST BROWN TODD, LLC, 2200 PNC CENTER, 201 E. FIFTH STREET, CINCINNATI, OH, 45202, US

(73) Assignee: Molecular Research Center, Inc., Cincinnati, OH (US)

(21) Appl. No.: 12/053,020

(22) Filed: Mar. 21, 2008

Related U.S. Application Data
(60) Provisional application No. 60/896,564, filed on Mar. 23, 2007.

(51) Int. Cl. A61K 38/22; A61K 31/65; A61K 31/60; A61K 31/56; A61K 33/32; A61K 33/26

(52) U.S. Cl. 424/641; 514/152; 424/646; 514/154; 514/165; 514/171; 514/2; 514/769; 514/420; 514/568; 514/569

(57) ABSTRACT

The present invention relates to an improved method for treating a wide range of inflammatory disorders by administering a tetracycline compound together with an effective inhibitor of tetracycline absorption, such as polyvalent metals. Pharmaceutical compositions used in that method are also taught.

This application is related to and claims priority from U.S. Provisional Patent Application No. 60/896,564, filed Mar. 23, 2007, incorporated herein by reference.

### BACKGROUND OF THE INVENTION

Inflammation is a localized response to trauma, toxins, neoplasia or microbial invasion and it is characterized by symptoms including redness, heat, swelling and pain. Inflammation comprises cellular, exudative and molecular components. The cellular component involves the movement of white blood cells from blood vessels into the inflamed tissue. The exudative component involves the movement of fluid containing proteins such as fibrin, cytokines and antibodies. The molecular component comprises a diverse series of molecules including: cytokines, prostaglandins, nitric oxide, immunoglobulins and cell adhesion molecules. Symptoms of inflammation at the molecular level include: 1) changes in cell components and metabolites comprising ion channels, cytokines, chemokines, receptors, cell adhesion molecules, innate binding molecules, transcription factors, and signal transduction molecules including nitric oxide; and 2) elevated levels of inflammatory markers in the circulation comprising: white blood cells, erythrocyte sedimentation rate, C-reactive protein, rheumatoid factor, immunoglobulin E, and cytokines including tumor necrosis factor alpha and interleukin 6.

Excessive inflammation or prolongation of the inflammatory process may result in inflammatory disorders and dysfunction of the organ involved. Inflammatory disorders include diseases as diverse as diabetes, obesity, atherosclerosis, viral diseases, cataracts, reperfusion injury, cancer and sarcoidosis; post-infectious meningitis and rheumatic fever; rheumatic diseases comprising systemic lupus erythematosus, osteoarthritis, rheumatoid arthritis; skin inflammatory disorders comprising various forms of acne and rosacea; and intestine inflammatory disorders including irritable bowel syndrome and Crohn's disease. The centrality of the inflammatory response in these varied disease processes makes its regulation a major element in the prevention, control or cure of human disease. A comprehensive review of inflammation published by Gallin et al. (1999) and additional examples of inflammation and inflammatory disorders in references: Hansson, 2005, Wellen, 2005, Karin, 2005, Popovic, 2005, U.S. Pat. Nos. 5,919,775 and 7,122,578, and US Published Patent Applications 2005/0164993 and 2006/0194773, are incorporated herein by reference to further define inflammation and inflammatory disorders. In addition, there is now extensive evidence that inflammation within the central nervous system (CNS) contributes to many acute and chronic degenerative disorders and perhaps some psychiatric diseases. The CNS disorders and diseases associated with inflammation comprise epilepsy, brain trauma, multiple sclerosis, Parkinson's disease and Alzheimer's disease (Lucas, 2006)

Pathogenesis of inflammation involves production of pro-inflammatory cytokines at the inflamed site. Production of the pro-inflammatory cytokines can be triggered by a local injury, local change in metabolic processes or by infection with bacteria and/or other microorganisms (Day, 2005; Golub, 2006). Based on these observations, most treatments of inflammatory disorders are focused on sites or tissues where the inflammatory changes are observed. Consequently, administration of anti-inflammatory drugs encompasses systemic and/or local delivery of a drug into the site of inflammation. For example, treatments of rheumatoid arthritis are based on delivery of anti-inflammatory drugs, via the circulation, to the affected sites where the inflammation actually appears (Gallin, 1999). Also, topical delivery of drugs is used in treatment of inflammation. For example, topical administration is used to treat skin inflammatory disorders such as rosacea and acne (U.S. Pat. No. 7,078,048).

Arthritis is one of the most studied inflammatory disorders. Despite numerous studies, the exact mechanisms that contribute to pathogenesis of the disorder are still largely unknown. It is generally accepted that specialized leukocytes and cells cooperating with leukocytes are implicated in pathogenesis and treatment of various forms of arthritis. A growing list of inflammatory cells and pro-inflammatory molecules includes various T cells, B cells, antigen presenting cells, including dendritic cells and the extensive list of pro-inflammatory cytokines such as TNFα and IL-1. A recent addition to this list is interleukin IL-32 implicated in pathogenesis of rheumatoid arthritis (Joosten, 2006).

Pharmacological agents used to treat inflammatory disorders include steroidal and non-steroidal compounds. Tetracyclines, the pharmacological agents used in the current invention, belong to the non-steroidal group.

Tetracyclines form a distinct class of antibiotics with pleiotropic activity in bacteria and mammalian cells. Tetracyclines are available as natural products, tetracycline semi-synthetic compounds and chemically modified tetracyclines (Chopra, 2001, U.S. Pat. No. 7,008,631, US Published Patent Application 2006/0194773). The most commonly used tetracyclines are natural (tetracycline and oxytetracycline), and semi-synthetic (doxycycline and minocycline). Synthetic tetracycline compounds are structurally related to the antibiotic tetracyclines but have their antibiotic activity substantially or completely eliminated.

In the antimicrobial action, tetracyclines act as broad-spectrum antibiotics that inhibit bacterial protein synthesis at the ribosomal level. The minimum inhibitory concentration (MIC) of tetracyclines causing growth arrest of bacteria strains is typically in the range of 0.1 μg/ml to 32 μg/ml. Petersen et al (1999) reports that tetracycline MIC for various strains including Escherichia coli, Staphylococcus aureus and Salmonella sp. is in the range of 0.12 μg/ml to 32 μg/ml, and for minocycline is in the range of 0.06 μg/ml to 32 μg/ml depending on the strain sensitivity. Webster et al (1982) reports that MIC for Propionobacterium acnes sensitive and insensitive strains is, respectively: 0.6 μg/ml and 5-10 μg/ml for tetracycline, and 0.3 μg/ml and 5 μg/ml for minocycline. Agwuch, 2006 reports that antibiotic MIC₅₀ for doxycycline in man is in the range of 0.1-25 μg/ml.

Antimicrobial action of tetracyclines is significantly inhibited by calcium and magnesium. D'Amato (1975) reports that MIC of tetracycline for Pseudomonas sp. increases up to 8-fold in the presence of 2.1 mM calcium or 1.4 mM magnesium salts, and up to 32-fold when both cations are present in a bacteria growth medium.

The tetracycline action in mammalian systems comprises effects on inflammation, proteolysis, angiogenesis, apoptosis and bone metabolism (Chopra, 2001, Roberts, 2003, Sapadin, 2006). The anti-inflammatory action of tetracyclines is of special interest as it has been linked to arthritis, cancer, asthma, cardiovascular disorders and skin disorders. In contrast to the anti-microbial action of tetracyclines, the underlying mechanism of anti-inflammatory action of theses compounds in mammalian cells is still elusive. Experimental data indicate that tetracyclines have effects on several processes related to inflammation. Some of these effects are observed in vitro at very high concentrations of tetracyclines; for example, at a level >20 μg/ml, a level that cannot be approached in the blood following a standard therapy. Typically, the peak concentration of tetracyclines in serum is in the range of 2-5 μg/ml (Agwuh, 2006).

It is not resolved to what extent the antiinflammatory action of tetracyclines is related to their antibiotic activity. For example, in acne, the presence of P. acnes appears to be associated with formation of inflammatory lesions and successful antibiotic treatment of acne is associated with a reduction in the P. acnes population. However, the density of P. acnes on the skin does not correlate with the degree of inflammation or severity of acne. Also, the magnitude of reduction in P. acnes counts following antibiotic therapy does not correlate with clinical efficacy.

US Patent Application 2006/0293290 attributes effectiveness of acne treatment with minocycline to the antibiotic effect of this drug.

It is proposed that tetracyclines mitigate inflammation in the affected sites by inhibition of phagocytosis, suppression of neutrophilic migration and chemotaxis, inhibition of T-lymphocyte activation, inhibition of phospholipase A2, inhibition of expression of nitric oxide synthase, inhibition of metalloproteinase activity, inhibition of secretion of pro-inflammatory cytokines and stimulation of secretion of anti-inflammatory cytokine (Dreno, 2004; Sapadin, 2006; US Published Patent Application 2006/0194773, paragraph 0060). The anti-inflammatory effects of tetracyclines are observed in the range from about 0.3 μg/ml to about 40 μg/ml (Krakauer, 2003, Amin, 1996, Kuzin, 2001, Golub et al., 1998). The effective concentration range for anti-inflammatory action of tetracyclines overlaps with the effective range for antibiotic action of these drugs.

The complexity of anti-inflammatory effects of tetracyclines has made it difficult to determine the underlying mechanism of their in vivo action. This is particularly true when these effects are observed in a variety of disorders with an inflammatory component, such as various forms of arthritis, skin disorders, autoimmuno and allergic disorders, cardiovascular disorders and cancer.

Following oral administration, absorption of tetracyclines into the blood occurs largely in the stomach and proximal small intestine. The absorption of tetracyclines is stimulated by chelating agents and surfactants, and inhibited by the presence of food, milk, and divalent or trivalent cations such as calcium, magnesium, zinc and iron. Absorption of tetracycline and oxytetracycline by food is reduced by about 50-70%. Inhibitory effect of food on absorption of doxycycline and minocycline is smaller and amounts to about a 20% to 30% reduction. Much higher inhibition of tetracycline absorption, about 85%, is caused by calcium and magnesium supplements and antacid compositions (Welling, 1977, Leyden, 1985, and Depperman, 1989).

Polyvalent cations form a complex with tetracycline compounds. Two calcium cations or one magnesium cation form a complex with one tetracycline molecule. The cellular uptake of metal-tetracycline complex is lower than the uptake of tetracycline compound alone. This results in inhibition of tetracycline absorption (Chopra, 2001). In treatments with oral administration, inhibition of tetracycline absorption and resulting lower concentration of these drugs in the blood is considered to be a factor negatively affecting anti-inflammatory action of tetracyclines.

To avoid inhibition of tetracycline absorption by food, a standard recommendation is to take a tetracycline compound one hour before or two hours after food consumption or administration of medicines or mineral supplements containing high level of divalent cations. These precautions are recommended for both antibacterial and anti-inflammatory treatments. As an extreme precaution to avoid food, U.S. Pat. No. 5,250,442 recommends fasting for 12 hours before oral administration of tetracycline in treatment of rheumatoid arthritis.

It is believed that serum level is an indication of the systemic effectiveness of tetracycline compounds as anti-inflammatory agents. Tetracycline levels achieved in serum after normal oral dosage (250-500 mg bid) are in the range of about 2 to 5 μg/ml. Most tetracyclines have to be given up to four times to maintain therapeutic concentration in serum (Chopra, 2001). A standard therapeutic dose of tetracyclines is in the range of about 250 mg-500 mg for tetracycline and about 100 mg-200 mg for semisynthetic tetracyclines.

Side effects of standard doses of tetracyclines include nausea, gastrointestinal irritation and dizziness. To alleviate these problems, therapeutic formulations were developed with lower doses of tetracyclines. Low dose of doxycycline (20 mg bid) was approved by the US Food and Drug Administration (FDA) for the treatment of adult periodontitis based on research indicating reductions in collagen, matrix degradation and decreased inflammation in gingival tissue (Golub, 1998 and DelRosso, 2004).

In a series of U.S. patents Ashley describes the use of low, non-antibiotic (subantimicrobial) doses of tetracycline compounds in treating acne and rosacea. U.S. Pat. No. 7,008,631 (Ashley, Mar. 7, 2006) describes methods of simultaneously treating ocular rosacea and acne, U.S. Pat. No. 7,014,858 (Ashley, Mar. 21, 2006) describes methods of treating acne and telangiectasia, and US Published Patent application 2005/0209202 (Ashley, Sep. 22, 2005) describes methods of treating rosacea. The activity and usefulness of tetracycline compounds are linked in these publications to the level of tetracyclines in the serum, and the route of tetracycline administration can be either oral or intravenous. In some cases topical application is considered. (see U.S. Pat. No. 7,008,631, column 9, line 40).

Low dose administration of doxycycline was recently approved by the FDA for treatment of inflammatory lesions (papules and pustules) of rosacea in adult patients. The formulation contains 30 mg immediate release and 10 mg delayed-release of doxycycline and is marketed by Collagenex Pharmaceuticals, Inc. under the trade name Oracea (NDA 50-805, 2006; U.S. Pat. Nos. 5,789,395 and 5,919,775). The 40 mg oral dose of immediate/sustained release doxycycline composition used in Oracea results in the antibacterial level of doxycycline in serum (Cₘₐₓ 0.6±0.2 μg/ml). In trials of Oracea, about 10% of subjects were evaluated as clear or almost clear, and the average decrease in the lesion counts was about 50%. Administration of a single dose of Oracea concomitant with food resulted in a decrease in the rate and extent of doxycycline absorption by 45% and 22%, respectively (Oracea, Prescription Information). The Prescription Information includes a warning that Oracea should be taken one hour before or two hours after meals.

It has been suggested that effects of low doses of doxycycline on inflammatory rosacea and acne may be due, in part, to the effect on inflammatory processes directly in the target site by inhibition of production of bacterial chemotactic factor, metalloproteinase activity and production of pro-inflammatory cytokines (Bikowski, 2003, Weinberg, 2004, U.S. Pat. No. 5,789,395).

The semi-synthetic tetracyclines such doxycycline and minocycline are more lipophilic than tetracycline. This allows for more effective cell penetration and absorption of semi-synthetic tetracyclines into the blood. The semi-synthetic tetracyclines are also less sensitive than natural tetracyclines to the inhibition of their absorption by polyvalent metals. These properties have made doxycycline and minocycline the tetracyclines of choice in the treatment of both bacterial infections and inflammation. In antiinflammatory treatments, the effective dose of doxycycline and minocycline is smaller than the effective dose of tetracycline.

Since 1971 tetracyclines have been used experimentally to treat arthritis. A review of these trials indicates that administration of tetracyclines for more than 3 months leads to modest reduction in disease activity and acute phase reactants in rheumatoid arthritis (RA). The beneficial effect was observed in trials with minocycline (Stone, 2003). Treatment of arthritis with tetracycline resulted in no significant benefit (Skinner, 1971). The daily doses of tetracyclines in these trials were: doxycycline 50 to 200 mg, minocycline 10 to 200 mg and tetracycline 250 mg.

Treatments of osteoarthritis have been focused on inhibition of inflammatory cytokine production and inhibition of metaloproteinases in the affected sites. A modest effect of doxycycline on contralateral, but not on index, knee was observed (Pelletier, 2006).

In another example, treatments of arthritis combine antibacterial action of tetracyclines with acyclovir, as an antiviral agent, and metronidazole or nitroimidazole, as antiprotozoal agents (U.S. Pat. No. 7,053,073 and US Published Patent Application 2006/0172956).

To date there is no established treatment of arthritis with tetracyclines. All treatments of arthritis using tetracyclines have been performed on an experimental basis.

For systemic delivery, tetracyclines are administered orally or parenterally including intravenous, intramuscular and subcutaneous injections. The most frequent is oral administration.

Tetracyclines can be administered orally in the form of tablets, pills, capsules and fluids. For example, doxycycline for oral administration is marketed by Pfizer Inc, NY, under the trademark Vibramycin. Vibramycin is indicated for the treatment or prevention of infectious diseases and prophylaxis of malaria. Vibramycin is available in various forms including: capsules (NDC 0069-0940-50 at fda.com) containing 50 or 100 mg doxycycline, tablets (NDC 0069-0990-50 at fda.com) containing 100 mg doxycycline, and coated tablets (Vibra-Tabs, NDC 0069-0990). These tablets provide sustained release of doxycycline. The release starts in the stomach after a 20-minute delay. Another form of Vibramycin is syrup (NDC 0069 B0871-93 at fda.com) containing 50 mg doxycycline as a calcium salt per 5 ml and inert ingredients comprising calcium chloride.

U.S. Pat. Nos. 4,126,680 and 4,081,527 describe liquid compositions containing calcium and magnesium salts of doxycycline, oxytetracycline and chlortetracycline wherein the metal to tetracycline compound molar ratio is from about 1:1 to about 2:1. U.S. Pat. No. 3,957,980 describes injectable solutions of doxycycline containing phosphates and magnesium salts wherein the molar proportion of metal to doxycycline ratio is up to 8:1. U.S. Pat. No. 3,275,513 describes oral compositions comprising tetracycline compounds, urea, alcohol and calcium salts wherein the calcium to tetracycline molar ratio is 2:1.

U.S. Pat. No. 2,736,725 discloses a water soluble complex composed of a tetracycline compound, aluminum, metal ion (including calcium and zinc ions), and alpha-hydroxy carboxylic acid wherein the maximal molar ratio is 1:8:10:24. A solution containing this complex has been used for intramuscular injection as an antibiotic. Since the '725 patent issued it has been established that aluminum is toxic to humans and soluble salts of this metal should not be incorporated into pharmaceutical formulations.

U.S. Pat. No. 4,060,605 discloses a water-soluble doxycycline derivative administered in the form of a salt with metals, including calcium and magnesium.

U.S. Pat. No. 4,061,676 indicates that calcium salts of tetracyclines can be used for preparation of stable oral suspensions (col. 4, par. 40).

A solid composition containing a doxycycline and metal complex has been described in the US Published Patent Application 2005/0019396. The doxycycline-metal complex includes mono- and bi-valent metals. The inventors hypothesize that the preferable ratio of metal to doxycycline is from 1.5 to 2.5. US Published Patent Application 2006/0183719 describes another solid composition with a tetracycline compound—metal complex. The metal part of the complex includes mono- and bi-valent metals. In the complex, the molar ratio of metal to tetracycline compound is from about 3:1 to about 1:3.

The compositions with metal and tetracycline compounds are designed for treating bacterial infection. Absorption of tetracyclines into the blood is the critical step for reaching infected sites in the anti-bacterial treatments. The '396 and '719 published applications disclose that amounts of metal salts used in compositions do not significantly interfere with absorption of tetracyclines. The '396 application discloses that a therapeutic amount of doxycycline can be absorbed from the doxycycline-calcium complex in a solid dosage form, where the calcium to doxycycline molar ratio is at maximum 3:1 (paragraphs 0021-0023). In the '719 application it is disclosed that the body can effectively absorb a tetracycline from a metal complex in a solid dosage form (paragraph 0030).

Compositions comprising tetracyclines and calcium sulfate were used for implants to secure long term (days) release of tetracyclines and other medications (U.S. Pat. Nos. 6,753,007 and 5,807,567)

To avoid gastrointestinal irritation and nausea, a small amount of food, milk and apple juice was allowed in cases were administration of semi-synthetic tetracyclines was causing gastrointestinal tract irritation and nausea (Vibramycin). Another approach to limit gastrointestinal irritation caused by tetracyclines in treatment of bacterial infections is the use of tetracycline salts with limited solubility. A review of these compositions is included in the U.S. Pat. No. 5,538,954, incorporated herein by reference.

U.S. Pat. No. 4,837,030 a describes controlled release composition containing minocycline. Minocycline is released partially in the stomach and partially in the intestine in order not to produce nausea or dizziness upon oral administration during antibacterial therapy.

U.S. Pat. No. 6,638,532 describes three dosage forms combining immediate release and delayed release compositions containing tetracycline and doxycycline, for treating bacterial infections.

Another product for oral administration containing a tetracycline compound is Minocin MR, indicated for the treatment of acne. Minocin MR capsules are a product of Wyeth (Wyeth, 2005) that contains 100 mg of minocycline hydrochloride in pellets. These capsules have been formulated as a “double delivery” system in which a portion of the minocycline dose is delivered in the stomach, and a second portion of the dose is available for absorption in the duodenum and upper GI tract (Wyeth, 2005, p. 5.2).

Enteric compositions have also been used to deliver magnesium (U.S. Pat. Nos. 6,887,492 and 4,150,111).

Pharmaceutical formulations of tetracycline comprise adjunct, non-active compounds including: carriers, binders and tableting agents that promote dissolution and delivery of a tetracycline compound into the gastrointestinal tract. Since polyvalent cations inhibit absorption of tetracyclines, pharmaceutical formulations comprising tetracyclines exclude significant amount of soluble salts of these cations. Small amounts of magnesium stearate or calcium stearate, less than 10 mg per dose, are added as wetting agents.

Inflammation is one of the major pathological states observed in a variety of disorders. The diverse outcome of the inflammatory disorders in terms of sites and pathology has one determinant in common. There is no clear explanation for the origin of these disorders. Prevailing theories refer to the multi-factorial origin of the inflammation. Currently, steroid and non-steroid pharmaceuticals are used for treatment of various processes involved in the inflammation (Day, 2005). They provide certain relief, but not a cure or satisfactory control of the disorder. With inflammation affecting millions of people, there is an urgent need to develop better treatment of inflammation. To successfully approach treatment of inflammation it is necessary to develop better understanding of the main contributing factor(s) triggering inflammation, and to develop a new treatment to significantly alleviate or cure inflammatory disorders.

All patents, patent applications and other references described in this application are incorporated herein by reference.

### SUMMARY OF THE INVENTION

The current invention describes a novel approach for the treatment of inflammatory disorders. The new treatments are based on the finding that processes occurring in the intestine critically affect inflammatory disorders in peripheral sites. As an example, it is disclosed that the antiinflammatory action of tetracycline compounds in peripheral sites originates in the small intestine, and that it is the critical step in the antiinflammatory action of these compounds. The effective compositions of the current invention comprise a tetracycline compound(s) and polyvalent metals as inhibitors of tetracycline transport from intestine into the blood. Contrary to previous assumptions, the tetracycline transport inhibitors facilitate effectiveness of tetracyclines in the anti-inflammatory treatments.

The compositions and methods of present invention comprise natural, semi-synthetic and synthetic tetracyclines, and pharmaceutically acceptable salts of tetracyclines.

Compositions of the present invention comprise a single tetracycline compound or a mix of tetracycline compounds.

Compositions of the current invention are administered orally, and can be used alone or in combination with other medications.

Therapeutic use of tetracycline compositions of the current invention comprise treating inflammatory disorders and disorders affected to a various degree by inflammation, including arthritis, cancer, diabetes, inflammatory skin disorders, cardiovascular disorders and CNS disorders.

### DETAILED DESCRIPTION OF THE INVENTION

The terms defined below are used in text, including claims, for the purpose of presenting the current invention:

Singular forms, also indicated by “a”, “an” and “the”, include plural referents unless the context clearly indicates otherwise. In some sentences in the text this is indicated by (s) added at the end of a word.

A “subject” or “patient” is a vertebrate, preferably a mammal and more preferably a human.

Effective compound, Effective amount, denotes a compound or amount of a compound(s) that is sufficient for a desirable change in at least one symptom of a disorder, and/or a desirable change in a biological process. The change due to the effective amount or effective compound should be at least about 15% of the value without the amount or compound.

Inflammation, is the response of living tissue to damage, trauma, toxins, neoplasia or microbial invasion, and it includes one or more of the following characteristics: redness, heat, swelling and pain. Inflammation comprises cellular, exudative and molecular components. A further definition, examples and symptoms associated with inflammation are included in the patent application and all relevant references in the text herein.

Inflammatory disorder, denotes a disorder with the inflammation as a main or adjunct component of the disorder. This term also includes a tetracycline responsive state wherein a pharmacologically active tetracycline compound cures, alleviates or prevents a disorder by other means than the antimicrobial action at the site of inflammation. A further definition, examples and symptoms associated with inflammation are included in the present application and all relevant references in the text of this herein.

Treating, treatment, comprise therapeutic and/or prophylactic treatment.

Treatment of inflammation or anti-inflammatory treatment, are intended to include curing, preventing, alleviating, or affecting in a desirable direction of at least one symptom associated or caused by inflammation and/or inflammatory disorder.

Anti-inflammatory action, comprises biological and/or biochemical action or activity of a pharmaceutical compound(s) during curing, preventing, alleviating, or affecting in a desirable direction of at least one symptom associated with or caused by inflammation and/or inflammatory disorder. This term does not include transport, and processes associated with transport, of said compound(s) to the site of its anti-inflammatory action.

Peripheral site, is intended to mean a site in the human or mammalian organism distal to the gastrointestinal tract.

Tetracycline compound(s), tetracyclines. These terms include compounds with a linear fused four hexacyclic-ring structure to which a variety of functional groups are attached. A representative structure for these compounds is tetracycline, with the following chemical structure, 4-(Dimethylamino)-1,4,4α,5,5α,6,11,12α-octahydro-3,6,10,12,12α-pentahydroxy-6-methyl-1,1′-dioxo-2-naphtacenecarobxamide (Merck Index 8913). Tetracycline compounds comprise natural, semi-synthetic and synthetic structures. Examples of tetracycline compounds are included in Chopra, 2001, US Published Patent Applications 2006/0166945 and 2006/0194773.

Absorption of tetracycline. Transport of a tetracycline compound from the gastrointestinal tract into the blood in humans and other mammals and vertebrates. A process of tetracycline absorption in other biological systems is described by added specific terms, for example, tetracycline absorption into bacterial cells.

Inhibitor of tetracycline absorption. A compound or mix of compounds capable of inhibiting, by at least 10%, transport of tetracyclines from the gastrointestinal tract into the blood. Compounds inhibiting release of tetracycline(s) from pharmaceutical compositions, such as enteric coating and delayed-release compounds, are not included in this definition.

Effective inhibitor of tetracycline absorption. This term comprises inhibitors of tetracycline absorption such as polyvalent metal cations, that are capable of inhibiting tetracycline absorption by at least about 15%, and not more than about 85%.

Effective amount of inhibitor of tetracycline absorption. This term describes an amount of polyvalent metal cations and other compounds inhibiting tetracycline absorption by at least about 15%, and not more than by about 85%.

Inhibitor of gastric absorption. A compound(s) capable of inhibiting, by at least 50%, transport of compounds such as tetracyclines from the stomach into the blood. Compounds inhibiting release of tetracycline(s) from pharmaceutical compositions, such as enteric coating and delayed-release compounds, are not included in this definition.

Intestine immunosystem. This term encompasses tissues, cells, and molecular components involved in the immunoresponse and located in intestine and mesenteric gland. The intestine immunosystem comprises specialized epithelial cells and lymphocytes localized in the intestine wall, Peyer's patches and mesenteric gland. The specialized cells and lymphocytes include dendritic cells, macrophages, neutrophiles, T cells including CD4+ T cells, CD8+ T cells and T helper cells, B cells, IgA- and IgE-producing cells, and microfold epithelial cells (M cells).

Gastric composition. An oral pharmaceutical composition formulated to release from 50% to 100% of its active component(s) in the stomach, and to release the remaining amount, if any, of active component(s) in the intestine.

Enteric composition. An oral pharmaceutical composition formulated to release from 0 to 50% of its active component(s) in the stomach, and after passing through the stomach, the remaining amount of active component(s) is released in the small intestine. Enteric compositions may contain smaller units (pellets) combined into a single oral composition. Enteric compositions comprise compositions with enteric coating and delayed release compositions, or combination thereof. Enteric compositions comprise compositions encapsulated in two or more layers providing for a controlled multi-step release of two or more active components. The multi-step release may occur in the intestine or partially in the stomach and partially in the intestine.

Enteric coating. A coating (coat) of an oral pharmaceutical composition that blocks, by at least 90%, gastric release of active component(s) of said composition. After passing through the stomach, the enteric coating releases active component(s) of said composition in the small intestine. Enteric coating may be used to coat a composition in the form of a single pill, tablet, capsule, or to coat smaller units (pellets) that can be combined into a single oral composition containing several smaller units.

Delayed release composition. An oral pharmaceutical composition formulated for a continuous release of its active component(s). The continuous release is initiated in the stomach and lasts for a period of at least 30 minutes. The delayed release compositions comprise compositions with two or more layers designed for a controlled multi-step release of ingredients, partially in the stomach and partially in the intestine.

% (percent). When % refers to the concentration of a compound in a solution, it is a percentage of weight of a compound per weight of a solvent used to dissolve said compound.

Treatments of inflammatory disorders are focused on sites affected by inflammation. These treatments are not sufficiently effective, and in some cases such osteoarthritis only surgical procedures can provide a significant, though only temporary, relief for patients. The current invention discloses that processes occurring in the intestine critically affect inflammatory disorders. This disclosure provides a basis new and more effective treatments of inflammatory disorders.

The anti-inflammatory compositions and methods of the current invention target the intestine, and more specifically the small intestine and small intestine immunosystem. Tetracyclines are shown as an example of compounds useful for treating inflammatory disorders using this new approach. By directing tetracycline action toward the intestine, the inflammatory disorders in the intestine and peripheral sites are effectively treated.

When administered orally, tetracycline compounds are absorbed into the blood in the stomach and in the proximal part of the small intestine. As reviewed in the Background section, absorption of tetracycline compounds from the gastrointestinal tract into the blood is inhibited by from about 85% to 15% by food and polyvalent metal cations. As a result, food and polyvalent cations increase retention of tetracyclines in the intestine and decrease the level of tetracyclines in the blood. In human subjects, administration of a tetracycline compound concurrently with a protein diet decreases serum tetracycline level to 1.8 μg/ml and doxycycline level to 2.6 μg/ml from the level of 4.4-4.7 μg/ml measured in fasting subjects (Welling, 1977). Administration of antacid (aluminum-magnesium hydroxide) decreases doxycycline serum level to 0.45 μg/ml from 2.7 μg/ml in fasting subjects (Depperman, 1989). The inhibition of tetracycline absorption has been considered as a negative effect of food and polyvalent cations on the anti-inflammatory action of tetracyclines.

The current invention discloses that, unexpectedly, food and polyvalent metal cations actually increase effectiveness of tetracyclines administered orally. It is disclosed that polyvalent metal salts can be utilized in compositions and methods of this invention to retain tetracyclines in the intestine and to facilitate effective anti-inflammatory treatments.

Compositions and methods of the current invention comprise natural, semi-synthetic and synthetic tetracyclines, and pharmaceutically acceptable salts of tetracyclines.

Compositions of the current invention comprise a single tetracycline compound or a mix of tetracycline compounds.

Compositions of the current invention are administered orally, and can be used alone or in combination with other medications.

A tetracycline compound effective in the current invention has the following chemical properties: 1) sufficient water solubility to exert effect on cells in aqueous environment, for example to approach concentration of 10 μg/ml; 2) ability to form complexes with polyvalent metal cations; and 3) its absorption from the gastrointestinal tract is inhibited by a polyvalent metal cation by at least about 15%.

In the current invention, polyvalent metal cations provide inhibition of absorption of tetracyclines. This results in accumulation of tetracyclines in the intestine, and more specifically in the small intestine. Polyvalent metal cations may also cooperate with tetracyclines by affecting cell metabolism. For example, it is well known that calcium cations play an important role in the intracellular signaling, including participation in signaling pathway in leukocyte T activation (Gallin, 1999). It is also known that calcium cations, through the calcium-sensing receptor, modulate proliferation and differentiation of intestinal epithelial cells (Herbert, 2004). In addition, low magnesium level in humans correlate with elevated C-reactive protein, a marker for inflammation (Guerro-Romero, 2002).

Accumulation of tetracyclines in the intestine can be further facilitated by the use of enteric compositions that prevent gastric release and release tetracycline compound in the small intestine.

In the current invention, an effective polyvalent metal cation provides the ability to: (1) form metal-tetracycline compound complex; (2) inhibit absorption of tetracyclines; and (3) increase effectiveness of anti-inflammatory action of tetracyclines.

Pharmacologically acceptable salts of di- or tri-valent metals, such as: chlorides, acetate, gluconate and carbonate salts of calcium, magnesium, zinc, and iron, can be used in the invention. Chelating anions, such as citrate or EDTA, decrease concentration of available metal cations and should not be used in compositions of the present invention.

Polyvalent metal salts can be a part of pharmaceutical compositions and/or foods. The daily dose of polyvalent metal salts in compositions of the invention should be preferably within the limit of the recommended daily allowance (RDA) for a specific metal. Current RDA calculated by the US Food and Drug Administration for calcium is 1 g and for magnesium is 0.4 g, and for all other polyvalent metals RDA is <20 mg. For example, RDA for iron, zinc, manganese, and copper is: 18 mg, 15 mg, 2 mg, and 2 mg, respectively.

For formulating compositions of the present invention, it is important to consider the RDA and potential toxicity of excess of metal in a daily diet. Due to the sufficiently high RDA, the effective polyvalent metal cations in the current invention are calcium and magnesium. The preferred anions are chlorides and carbonates. The most preferred cation is calcium and the most preferred salts in the current invention are calcium carbonate and calcium chloride. Calcium compositions of the invention can be supplemented with other polyvalent metal compounds.

The most preferred compositions of this invention comprise calcium and magnesium salts. It is known that calcium oral supplements cause constipation. The addition of magnesium to the calcium-containing oral compositions counteracts this calcium effect. In the current invention, the preferred molar ratio of calcium to magnesium is from about 1:1 to about 3:1.

Preferentially, the tetracycline-calcium compositions of the current invention are administered together with a meal.

Calcium is a common food component of plant and animal origin. The amount of food-derived calcium in a diet varies significantly. The rate of calcium, absorption depends on solubility of calcium in the stomach. The soluble calcium is absorbed mostly in the stomach. Fordtran, 1966 reported that after eating a diet with a higher level of soluble calcium, such as a milk-doughnut meal, the concentration of dissolved calcium in man's stomach is about 20-30 mM, and decreases to 3-5 mM when the digested food reaches the ileum. For comparison, after eating a steak meal, concentration of dissolved calcium in man's stomach is approximately 3-4 mM and it remains at this level when the meal passes through the small intestine.

Aqueous compositions comprising tetracycline and dissolved polyvalent metal salt(s) are not preferred in the current invention due to the rapid absorption of dissolved metal cations in the stomach.

In compositions of the current invention, an effective amount polyvalent metal salt(s) comprises more than about a 10-fold higher molar amount, and preferentially about a 15-fold higher molar amount of polyvalent metal(s) than the molar amount of the tetracycline. Typically, the preferred upper limit for this ratio is about 40-fold higher molar amount of a polyvalent metal(s). The dose of polyvalent metal(s) above the 40-fold higher amount can be used in compositions containing small amount of tetracycline (40-60 mg), to provide sufficient amount of polyvalent metal cations in stomach and intestine, and to compensate for consumption of food (for example acidic fruits) or food additives containing high level of chelating agents and other compounds enhancing absorption of tetracyclines.

U.S. Pat. No. 2,806,789 discloses that the rate of tetracycline absorption can be augmented by calcium sequestering by material present in food such as ethylene tetraacetic acid, citric acid, ascorbic acid, polyphosphoric acid and pyrophosphoric acids and salts of these compounds. Additional chelating agents in plants are phenols and polyphenols. Also, it is known that phosphates enhance the intestinal absorption of tetracyclines, probably by precipitating interacting metallic ions (Sompolinsky, 1972). Other compounds increasing absorption of tetracyclines are surfactants.

Compounds that increase tetracycline absorption should be present either in small amounts or not included in compositions of the present invention.

Treatments of inflammatory disorders in the current invention comprise the use of natural, semisynthetic and synthetic tetracycline compounds. As indicated in the Background section, some tetracyclines, such as tetracycline and oxytetracycline, are more sensitive to the inhibitory action of polyvalent cations than semi-synthetic tetracyclines, such as minocycline and doxycycline. This property makes tetracycline and oxytetracycline the preferred tetracycline compounds in the current invention. The most preferred compound is tetracycline and the most preferred salt is tetracycline hydrochloride.

The most preferred compounds tetracycline, oxytetracycline as well as another preferred compound, chlortetracycline, have fewer side effects when compared with semisynthetic tetracyclines and chemically modified non-antimicrobial tetracyclines (CMT) such as doxycycline, minocycline and CMT-3. Doxycycline, minocycline and CMT-3 inhibit proliferation of various cell types in vitro and in vivo (Banack, 1979, Lokeshwar, 1998, and Bendeck, 2002). The intestine epithelial cells have a high proliferation rate. Thus, the anti-proliferating effect of semisynthetic or synthetic tetracyclines may lead to potential side effects, especially during a long-term therapy. In our tests with the human intestine cell line CCL-221, doxycycline (5 μg/ml) inhibited cell growth by about 60%, while no inhibition of cell growth was observed with tetracycline (5 μg/ml). In experiments with the human lymphoma Molt-4 cells, doxycycline (5 μg/ml) inhibited by about 80%, and tetracycline (5 μg/ml) only by about 20% growth of these cells.

In addition, prolonged treatment with minocycline (for acne) has been shown to induce the syndrome described as “drug-induced lupus”.

### Gastric Compositions.

Gastric compositions of the current invention are formulated for release of a tetracycline compound(s) in the stomach, concurrently with polyvalent metal salt(s).

Preferred gastric compositions of the current invention comprise effective amount of a tetracycline compound in the range from about 1 Emole to about 15 μmoles per kg of body weight, per single dose. The higher doses of tetracyclines are effective but may have toxic side effects.

For administration with a meal and without a calcium additive, or with a calcium additive but without a meal, the preferred range of a tetracycline compound(s) is from about 0.5 μmoles to about 10 μmoles per kg body weight, per single dose.

For administration with a meal and with a calcium additive, the preferred single dose of a tetracycline compound(s) is from about 2 μmoles to about 5μ moles per kg body weight. This corresponds to the dose of about 67 mg to about 168 mg of tetracycline hydrochloride per 70 kg body weight.

Polyvalent metal salts used in the gastric compositions should be soluble in water at pH ranging from about pH 1 to about pH 8.5, and dissociate to cations capable of forming a complex with tetracycline compounds. The preferred polyvalent metal salts are chlorides. Other salts, such as acetates and gluconates, can also be used. The compositions with calcium and/or magnesium salts can be supplemented with pharmaceutically acceptable amounts of zinc and iron salts, and polyvalent metal microelements.

Some water insoluble or sparsely soluble salts or compounds that can be used in the present invention react in the gastric acidic environment to form water-soluble salts. For example, practically insoluble in water calcium carbonate reacts in the stomach with HCl and converts to highly soluble in water calcium chloride. The dissociated calcium chloride provides calcium ions to form a complex with a tetracycline compound both at acidic pH in the stomach, and at neutral to alkaline pH in the intestine. An example of a sparsely soluble in water compound that can be used in the invention is magnesium oxide. Magnesium oxide combines in the stomach with water and forms magnesium hydroxide, converted in the stomach at acidic pH to magnesium chloride.

Examples of salts not useful in the current invention are calcium phosphate and calcium sulfate hemihydrate. Calcium sulfate hemihydrate (dried gypsum) is not soluble in water. Calcium phosphate can be, at least partially, dissolved in the stomach at acidic pH, but at neutral pH in the intestine calcium phosphate becomes water insoluble and it is not effective source of calcium ions. Also, it is known that phosphates promote intestinal absorption of tetracyclines.

The gastric compositions of the present invention comprise polyvalent metal salt(s) in an amount from about 10 μmoles to about 150 μmoles per kg body weight, per single dose. This range corresponds, for example, to 28-420 mg of calcium per single dose, for a 70 kg subject. The preferred amount is in the range of from 18-57 μmoles of polyvalent metal salt(s) per kg of body weight, per single dose. This corresponds to 50 mg-160 mg of calcium per single dose, for a 70 kg person. This amount of calcium is safely within the 1 g RDA for calcium.

In the current invention, the polyvalent metal additive can be consumed as a part of the tetracycline composition and/or it can be consumed as a separate composition. If consumed separately, the polyvalent metal additive should be consumed within about ±20 minutes of tetracycline compound administration.

In gastric compositions of the present invention, preferably the total daily dose of a metal should not exceed the RDA by more than 3 times. More preferably, the daily dose of polyvalent metals present in compositions of the present invention should not exceed RDA for a specific metal.

In the gastric compositions, the amount of calcium below 10 μmoles/kg/dose, taken together with food or alone, does not significantly contribute to effectiveness of tetracyclines.

Examples of gastric compositions include pills, tablets or capsules comprising: 1) 150 mg tetracycline hydrochloride, 300 mg calcium carbonate, 50 mg calcium chloride dihydrate and 100 mg magnesium oxide; 2) 80 mg doxycycline, 200 mg calcium carbonate, 50 mg calcium chloride and 75 mg magnesium oxide; and 3) 40 mg tetracycline hydrochloride and 150 mg calcium carbonate, 50 mg calcium chloride and 75 mg magnesium oxide.

### Enteric Compositions.

Enteric compositions of the current invention comprise delayed-release compositions and enteric coating compositions. Enteric compositions are widely used and known to those skilled in art. A review of enteric compositions is included in U.S. Pat. No. 6,887,492 (par. 40), incorporated herein by reference. Enteric coatings are made of non-toxic, edible polymers that are insoluble in gastric juice of the stomach. The enteric coatings have a dissolution point at pH>5 to resist the acidic environment of the stomach and to be dissolved at the proximal intestine.

Enteric compositions of the current invention deliver a tetracycline compound(s) to the small intestine together with polyvalent metal salt(s). Tetracycline compositions delivered to the intestine without polyvalent metal salts are less effective in the present invention.

The preferred enteric compositions of the present invention are coated with Eudragit 100-55 (Rohm GmbH, Germany), a methacrylic polymer. Eudragit L100-55 coating is substantially insoluble under the pH conditions prevailing in stomach (pH 2-pH 4), but soluble at pH above 5.5, prevailing in the small intestine.

The minimal effective amount of tetracycline compound in the enteric compositions with polyvalent metal salt(s) is 0.3 μmole per kg of body weight, per single dose. Preferentially, the upper limit for a tetracycline compound is 5 μmole per kg of body weight, per single dose. The higher doses of tetracyclines are effective but may have toxic side effects. The preferred range is from about 0.5 μmole to about 2.5 μmoles per kg body weight. This corresponds to the range of about 17 mg to about 84 mg of tetracycline hydrochloride per 70 kg body weight/dose.

The effective amount of polyvalent metal salt(s) in enteric compositions of the present invention is from about 4 μmoles to about 80 μmoles per kg body weight, per single dose. The preferred effective amount is in the range form 10-50 μmoles per kg of body weight, per single dose. This preferred amount corresponds to 28 mg-140 mg of calcium per single dose for a 70 kg subject.

Examples of enteric compositions comprise pills or tablets containing: 1) 75 mg tetracycline hydrochloride, 200 mg calcium carbonate, 50 mg of calcium chloride and 50 mg magnesium chloride; and 2) 40 mg tetracycline hydrochloride and 200 mg calcium carbonate, 50 mg calcium chloride and 50 mg magnesium chloride.

Calcium carbonate is practically insoluble in water. However, calcium cations from calcium carbonate can solubilize by chelating compounds. In our tests, an aqueous suspension of 7.5 mg of calcium carbonate per ml (75 mM) was completely solubilized, at pH 7.5, by the addition of an equimolar amount of sodium EDTA or the three-fold molar excess of sodium citrate. Thus, like in gastric compositions, calcium carbonate is used in enteric compositions to counteract sequestering of polyvalent metals, especially by components of plant food. In the enteric compositions of the current invention, calcium carbonate acts as a calcium-buffering compound. An increase in chelating capacity in consumed food is neutralized by solubilized calcium cations derived form calcium carbonate. An excess of calcium remains insoluble and passes through the intestine. An added advantage of the use of calcium carbonate is that carbonate ions are critical for antibacterial activity of antimicrobial peptides present in the intestine (Dorschner, 2006).

The amount of tetracycline compound(s) that preferentially can be used in the enteric compositions without polyvalent metals is from about 2 moles to about 8 moles per kg body weight.

The gastric release and enteric release compositions of the present invention can be administered one, twice or three times a day, preferentially with meals. If necessary, they can be administered four times a day. Preferentially, compositions of the current invention can be administered two or three times a day during the first 4 to 8 weeks of treatment followed by once a day administration during the next 3 to 6 weeks, as a the maintenance dose in the treatment. The preferred maintenance doses for a 70 kg subject are for the gastric compositions 50 mg to 40 mg of a tetracycline compound and for enteric compositions 30 to 20 mg of a tetracycline compound. Examples of the maintenance doses are provided in Examples 4 and 8.

The daily doses of tetracycline compound and treatment regimen of the present invention depend on the severity of inflammation. For a 70 kg subject with advanced inflammatory disorder, a preferred dose is >100 mg of a tetracycline compound in a gastric composition, or >60 mg in an enteric composition, administered 3 times a day for 1 week, and 2 times a day for the next 4-7 weeks. For a 70 kg subject with a mild inflammatory disorder, a preferred dose for an effective treatment is <100 mg of a tetracycline compound in a gastric composition or <60 mg in an enteric composition, is administered 2 times a day for 4-6 weeks.

In one embodiment of this invention, a 70 kg subject with mild rosacea (less than facial 10 papulo-pustules) is given 2 pills daily, each containing 75 mg of a tetracycline compound in gastric composition. For severe rosacea, 2 pills are administered daily, each containing 160 mg of tetracycline compound in gastric composition.

Compositions of the current invention with a tetracycline compound(s) can be formed as pills, tablets, capsules and other forms of pharmaceutical compositions. In addition to tetracycline compound(s) and polyvalent metal(s), the compositions can comprise other pharmaceutically active compounds and non-active excipients.

Pharmaceutically active compounds that can be included as adjunct active components in compositions of the current invention include, for example, antibiotics, steroid and non-steroid anti-inflammatory drugs, immunosuppressive drugs and hormones. Examples of adjunct active compounds include erythromycin, hydrocortisone, aspirin, cyclosporine, hydrocortisone, methotrexate and cytokine IL-10.

Other adjunct active components comprise health supplements, vitamins and microelements. The adjunct active components can be a part of single-stage release compositions for the release in the stomach or in the intestine, or multi-stage release compositions that release their components in part in the stomach and in part in the intestine.

Methods of the current invention comprise administration of the adjunct active components as separate compositions that can be administered concurrently or at different times than compositions of the present invention.

The non-active components of compositions of the current invention comprise excipients, carriers, binders and tableting agents to promote dissolution and delivery of a tetracycline compound into the small intestine. Compositions of the current invention comprise calcium chloride and magnesium chloride added as a source of metal cations and as dissolution agents. Preferably, only small amounts of surfactant compounds, such as wetting agents magnesium stearate or calcium stearate (less than 25 mg per dose), are added to compositions of this invention. Surfactants are known to facilitate absorption of tetracyclines.

The current invention encompasses a method for the effective use of anti-inflammatory drugs by targeting the intestine and intestine immunosystem for treatment of inflammatory disorders in peripheral sites. Without being limited by the example, the new method is exemplified by the use of anti-inflammatory compositions comprising tetracycline(s) and polyvalent metal(s). The anti-inflammatory effect of tetracycline-polyvalent metal compositions of the current invention results in a substantial reduction or elimination of inflammatory disorders. Inflammatory disorders are one of the most frequent diseases affecting human and animal populations. Examples of inflammatory diseases include: diabetes, obesity, atherosclerosis, cataracts, reperfusion injury, cancer, sarcoidosis, postinfectious meningitis, rheumatic fewer, rheumatic diseases comprising systemic lupus erythematosus, osteoarthritis and rheumatoid arthritis, skin diseases comprising various forms of acne and rosacea, autoimmune encephalitis, uveitis, thyroiditis, myasthenia, plus non-autoimmune diseases, such as asthma, allergy, colitis and stroke, and intestine inflammatory disorders including irritable bowel syndrome and Crohn's disease, and CNS disorders including epilepsy, brain trauma, multiple sclerosis, Parkinson's disease and Alzheimer's disease. Additional examples of inflammatory disorders are included in references: Gallin, 1999, Hansson, 2005, Wellen, 2005, Karin, 2005, 2005, Popovic, 2005, U.S. Pat. No. 7,122,578 and US Published Patent Application 2006/0194773.

The compositions and methods of the current invention allow for more effective treatment of inflammation and inflammatory disorders. For example, subjects with arthritis and osteoarthritis after taking a composition of Example 1 (150 mg tetracycline) for 6 weeks can find significantly alleviated disease symptoms, as well as alleviated pain and discomfort. Subjects with inflammatory rosacea after 4 weeks of taking the composition of Example 5 can be cleared from all facial symptoms of this disease.

In another embodiment of this invention, subjects with rheumatoid arthritis taking, twice a day, a pill comprising 150 mg of tetracycline hydrochloride described in Example 1, significantly alleviate the disease as measured by patient and physician global assessment including swelling and tenderness of joints, erythrocyte sedimentation rate, and patient's assessment of pain. Similar beneficial effects of the tetracycline-polyvalent metal treatment of the present invention are observed in treatment of osteoarthritis.

As another advantage, compositions of the present invention are not irritating to the gastrointestinal tract. Elimination of these gastrointestinal side effects is due to the presence of polyvalent metals in compositions of the invention.

Another advantage of the current invention is that tetracycline compositions and meals are taken at the same time. This contributes to lack of gastrointestinal irritation. Also, there is no need to wait one to two hours between meals and tetracycline uptake. This is especially convenient in the morning hours. If needed, compositions of the invention can be administered without a meal. The flexibility of administration regimen in the current invention increases subject compliance with the treatment.

Prolonged use of antibiotics changes the microflora of the gastrointestinal tract. This side effect is minimized in the treatments of present invention. The presence of polyvalent metals, and especially calcium and magnesium, diminishes the antibacterial effect of tetracyclines on commensal bacteria in the intestine. For some bacteria, tetracycline is over 30-fold less effective in the presence of calcium and magnesium salts (D'Amato, 1975). In the presence of calcium and magnesium, subantimicrobial concentrations of tetracycline for sensitive bacteria strains are much higher than anticipiated, for example in U.S. Pat. Nos. 7,008,631 and 7,014,858.

In addition, the compositions and methods of the present invention comprise the use of menthol. It is disclosed here that menthol alleviates side effects such as excess of gastrointestinal gases and bloating observed in some subjects taking the compositions of this invention. Menthol is a known carminative. In a novel application, menthol is used in this invention to prevent production of gases in the intestine. A minimal effective amount of menthol is 3 mg single dose per day, and the amount is in the range of about 4 mg to about 8 mg/dose/day. Menthol can be a part of compositions of the current invention or administered independently as a separate composition. Menthol can be also useful to alleviate discomfort related to gases and bloating induced by various agents and treatments, not limited to compositions and treatments of this invention.

Without being limited by the proposed mechanism, it is postulated that the therapeutic effect that tetracyclines has on inflammatory disorders in peripheral sites is realized by the effect of tetracyclines on interaction of the intestine immunosystem with food components and/or bacteria. It is proposed that the anti-inflammatory action of a tetracycline compound(s) affects the function of specialized leukocytes and cooperating cells of the intestine immunosystem.

It is known that food components and bacteria constitute an antigenic load in the intestine. The consequences of oral antigen administration include development of systemic and/or local immunological tolerance for a harmful antigen (oral tolerance). In oral tolerance, cell mediated and humoral (IgE, IgG and IgM) responses become tolerated. In tests of oral tolerance and autoimmune disorders, excessive amounts of protein antigens, such as collagen and ovoalbumin, were administered orally. The results of these tests showed only a modest suppression of mammalian autoimmune diseases including arthritis, type 1 diabetes, and models of Alzheimer's disease (Worbs, 2006, Faria, 2005; Toussirot, 2002; U.S. Pat. No. 6,010,722). To date, no therapy based on these findings is practiced.

The present invention discloses another food-induced phenomenon. A prolonged, excessive uptake of certain food components is not therapeutic but may lead to pro-inflammatory changes. This can be observed, for example, in people suffering from inflammatory rosacea. A person on a daily diet comprising about 150 g of tomato paste develops after 3 weeks of this diet a permanent sensitivity toward tomato products. In another example, a two-month excessive coffee uptake (up to 6 cups a day) sensitizes this person to coffee. In both cases the acquired sensitivity has appeared as inflammatory changes in skin in form of papules and pustules, in response to consumption of triggering such as tomato products or coffee. These observations put in questions the advantage of prolonged and administration of excessive amounts of collagen and ovoalbumin. It is disclosed in the present invention that the food-induced inflammatory changes can be blocked by tetracycline compositions, and preferentially by tetracycline-polyvalent metal compositions. A daily dose for blocking the food-induced rosacea is 50 mg-150 mg of tetracycline or doxycycline in compositions comprising calcium and magnesium (Examples 1-3 and 5-7).

It is disclosed here that a tetracycline compound(s) blocks pathological response of the intestine immunosystem to the food challenge, and, as a result, it alleviates inflammatory disorders in peripheral sites. The action of tetracyclines disclosed in this invention can be described as induction of tolerance of intestine immunosystem to pro-inflammatory food components, in subjects with an inflammatory disorder. It is also disclosed here that inflammatory disorders with pathological symptoms in peripheral sites are in fact disorders of the intestine immunosystem and should be treated us such. In this mechanism, various pathological agents and microorganisms detected in inflamed peripheral sites are secondary factors that contribute to an inflammatory disorder. Treatments focused on peripheral sites may alleviate but not substantially suppress or cure a disorder critically affected by the intestine immunosystem.

Immunological activity of the intestine is frequently under-appreciated. But this is an organ with the most abundant lymphoid tissue in the body. The human intestine contains 1012 lymphoid cells per meter (Faria, 2005). The intestine lymphoid tissue is located in Peyer's patches and the mesenteric gland and specialized lymphocytes are located in the intestine wall. A pathological response of the intestine immunosystem to certain food components may result in accelerated accumulation of activated B and/or T cells, and increase in level IgG, IgE and/or IgM in blood and in target sites, for example skin in rosacea or joints in rheumatic arthritis. In healthy subjects, a small pathological change in the skin induced by UV, or a bacterial infection in joints evokes normal immunoresponse and the change is alleviated. In subjects with over-responsive immunosystem, like in rosacea or rheumatoid arthritis, a similar change leads to the increased accumulation of leukocytes, swelling and other symptoms of inflammation in affected sites. The antibiotic treatment can bring some relief to a subject by alleviating inflammatory processes in a site of inflammation. However, the underlying cause of inflammation, the over-reactive intestine immunosystem can be effectively treated by targeting anti-inflammatory compounds, such as tetracyclines, to the intestine.

The controlling role of the intestine in inflammation explains the lack of success in trials aimed to improve effectiveness of tetracyclines by substituting intravenous injection for oral administration, as in trials described by Pillemer, 2003 and St. Clair, 2001. As indicated in the Background section, the intravenous administration of tetracyclines is frequently the recommended route of delivery of these drugs as anti-inflammatory agents. Another example is U.S. Pat. No. 5,919,775 (column 10, par. 10). The intravenous injection significantly limits the exposure of intestine to tetracyclines and thus, in accord with the present invention, diminishes the antiinflammatory effect of these drugs. A lack of understanding of pathogenesis of inflammatory disorders has prevented elaboration of more effective anti-inflammatory therapies and has exposed patients to unnecessary treatments.

The current invention describes the anti-inflammatory action of tetracyclines. It does not relate to the use of these compounds in treatments of infectious disorders caused by bacteria and microorganisms. Treatments of infectious disorders may require either oral or intravenous administration of tetracyclines without concurrent administration of polyvalent metal composition.

### EXAMPLES

The gastric and enteric pill compositions of the invention described in the examples are administered 2-3 times a day, preferentially with meals (breakfast, lunch and dinner). After 4-8 weeks of administration of these compositions, inflammatory disorders are significantly alleviated.

Components of compositions listed in the Examples below are formulated as pills in accordance with US FDA regulations. USP, NF grade components are obtained from Spectrum, Gardena, Calif.; Eudragit L100 55 is obtained from Rhom GmbH. KG, Darmstadt, Germany. Enteric pill compositions are coated using 3 mg of Eudragit L100 55 per cm² of the pill surface.

### Example 1

Gastric pill composition. 150 mg tetracycline hydrochloride, 300 mg CaCO₃, 50 mg CaCl₂ dihydrate, 100 mg magnesium oxide, 5 mg croscarmellose, 15 mg stearic acid, 5 mg magnesium stearate.

### Example 2

Gastric pill composition. 80 mg doxycycline hyclate, 200 mg CaCO₃, 50 mg CaCl₂ dihydrate, 75 mg magnesium oxide, 5 mg croscarmellose, 15 mg stearic acid, 5 mg magnesium stearate.

### Example 3

Gastric pill composition. 50 mg doxycycline hyclate, 150 mg calcium carbonate; 50 mg CaCl₂ dihydrate, 75 mg magnesium oxide, 5 mg croscarmellose, 15 mg stearic acid, 5 mg magnesium stearate.

### Example 4

Gastric maintenance pill composition. 40 mg tetracycline hydrochloride, 150 mg calcium carbonate; 50 mg CaCl₂ dihydrate, 75 mg magnesium oxide, 5 mg croscarmellose, 15 mg stearic acid, 5 mg magnesium stearate.

### Example 5

Enteric pill composition. 75 mg tetracycline hydrochloride, 200 mg CaCO₃, 50 mg CaCl₂ dihydrate, 50 mg magnesium chloride, 50 mg microcrystalline cellulose, 4 mg croscarmellose, 12 mg stearic acid, 4 mg magnesium stearate.

### Example 6

Enteric pill composition. 40 mg tetracycline hydrochloride, 200 mg CaCO₃, 50 mg CaCl₂ dihydrate, 50 mg magnesium chloride, 85 mg microcrystalline cellulose, 4 mg croscarmellose, 12 mg stearic acid, 4 mg magnesium stearate.

### Example 7

Enteric pill composition. 40 mg doxycycline hyclate, 200 mg CaCO₃, 50 mg CaCl₂ dihydrate, 50 mg magnesium chloride, 85 mg microcrystalline cellulose, 4 mg croscarmellose, 12 mg stearic acid, 4 mg magnesium stearate.

### Example 8

Enteric maintenance pill composition. 30 mg tetracycline hydrochloride, 200 mg CaCO₃, 50 mg CaCl₂ dihydrate, 50 mg magnesium chloride, 50 mg microcrystalline cellulose, 4 mg croscarmellose, 12 mg stearic acid, 4 mg magnesium stearate.

### REFERENCES

Agwuh K. N., MacGowan A., Pharmacokinetics and Parmacodynamics of the Tetracyclines Including Glycylcyclines, J. Antimicrob. Chemother., 58, 256-265, 2006.
Amin R. A. et al., A Novel mechanism of Action of Tetracyclines: Effects on Nitric Oxide Synthases, Proc. Natl. Acad. Sci., 93, 14014-14019, 1996.
Banack G. and Forsgren, Antibiotics and Suppression of Lymphocyte Function in Vitro, Antimicrob. Agents Chemother., 16, 554-560, 1979.
Bikowski J B, Subantimicrobial Dose of Doxycycline for Acne and Rosacea, SkinMed 2, 234-245, 2003.
Bendeck M. P. et al, Doxycycline Modulates Smooth Muscle Cell Growth, Migration, and Matrix Remodeling after Arterial Injury, Am. J. Pathol. 160, 1089-1095.
Chopra I and Roberts M, Tetracycline Antibiotics: Mode of Action, Applications, Molecular Biology, and Epidemiology of Bacterial Resistance, Microb. Molec. Biol. Rev. 65, 232-260, 2001.
D'Amato R. F. et al. Effect of Calcium and Magnesium on the Susceptibility of Pseudomonas Species to tetracycline, Gentamycin, Polymyxin b, and Carbenicilin, Antimicrob. Agents Chemother. 7, 596-600,1975.
Day R. O. et al., eds. Antirheumatic Therapy: Actions and Outcomes, Brikhauser Verlag, 2005, Basel, Switzerland.
DelRosso J. Q. A Status report on the Use of Subantimicrobial-Dose Doxycycline: A Review of the Biologic and Antimicrobial Effects of Tetracyclines, Cutis, 74, 118-122,2004
Deppermann et al., Influence of Ranitidine, piperazine, and Aluminum Magnesium Hydroxide on the Bioavailability of Various Antibiotics, Antimicrob. Agents and Chemother. 33, 1901 B 1907, 1989.
Dorschner R. A. et al. The Mammalian Ionic Environment Dictates Microbial Susceptibility to Antimicrobial Defense Peptides, FASEB J., 20, 35-42, 2006.
Dreno B et al., European Recommendation on the Oral Antibiotics for Acne, Eur, J. Dermatol. 14, 391-399, 2004.
Dougherty J. M. et al, The Role of Tetracycline in Chronic Bleparitis, Invest. Ophatomol. Vis. Sci. 32, 2970-2975, 1991.

Faria A. M. and Weiner H. L., Oral Tolerance, Immunol. Rev. 206, 232-259,2005.
Fordtran J. S. and Locklear T. W., Ionic Constituents and Osmolality of Gastric and Small-Intestinal Fluids after Eating, New Series, 11, 503-521, 1966.
Gallin J. I et al., eds, Inflammation, Basic Principles and Clinical Correlates, 3^{rd} ed., 1999. Lippincott Williams & Wilkins, Philadelphia.
Golub L. M. et al, Tetracyclines Inhibit Connective Tissue Brakdown by Multiple Non-Antimicrobial Mechanisms, Adv. Dent. Res. 12, 12-26,1998.
Golub, L. M. et al., Can Systemic Diseases Co-induce (Not Just Exacerbate) Periodontitis? A Hypothetical “Two-hit Model, J. Dent. Res. 85, 102-105, 2006.
Guerrero-Romero F. and Rodriges-Moran M, Relationship Between Serum Magnesium Levels and C-reactive Protein concentration, Int. J. Obesity 26, 469-474, 2002.
Hansson G. Inflammation, Atherosclerosis, and Coronary Artery Disease. N. Engl. J. Med. 352, 1685-1689, 2005.
Herbert S. C. et al., Functions and Roles of the Extracellular Ca²⁺-sensing Receptor in the Gastrointestinal Tract, Cell calcium 35, 239-247, 2004.
Joosten L. A et al., IL-32, A Proinflammatory Cytokine in Rheumatoid Arthritis, Proc. Natl. Acad. Sci., 103, 3298-3303, 2006
Karin M. and Greten F. NF-kB: Linking Inflammation and Immunity to Cancer Development and Progression. Nature Rev. Immunol. 5, 749-759, 2005.
Krakauer T. and Buckley M. Doxycycline Is Anti-Inflammatory and Inhibits Staphylococcal Exotoxin-Induced Cytokines and Chemokines, Antimicrob. Agents Chemother. 47, 3630-3633, 2003.
Kuzin I. I. et al, Tetracyclines Inhibit Activated B Cell Function, Intern. Immunol., 12, 921-931,2001.
Leyden J. J., Absorption of Minocycline Hydrochloride and Tetracycline Hydrochloride, J. Am. Acad. Dermatol, 12,308-312,1985.
Lokeshwar B. L. et al., Potential Application of Chemically Modified Non-Antimicrobial Tetracycline (CMT-3) Against Metastatic Prostate Cancer, Adv. Dent. Res. 12, 97-102, 1998.
Lucas S. M. et al. The Role of Inflammation in CNS Injury and Disease, Brit. J. Pharmacol. 147, S232-S240.
Oracea, NDA 050805, www.accessdata.fda.gov
Oracea Prescription Information
Pelletier J P. et al. Most Recent Developments in Strategies to Reduce the Progression of Structural Changes in Osteoarthritis, Arthritis Res. Ther. 206, 8, 2006 (arthritis-research.com/content/8/2/206.
Petersen et al. In Vitro and In Vivo Antibacterial Activities of a Novel Glycylglycine, the 9-t-Butylglycylamido Derivative of Minocycline (GAR-936), Antimicrob. Agents Chemother. 43, 738-74, 1999.
Pillemenr S. et al., Pilot Clinical trial of Intravenous Doxycycline Versus Placebo for Rheumatoid Arthritis, J. Rheumatol 30, 41-43, 2003.
Popovic n et al., Inhibition of Autoimmune Encephalomyelitis by a Tetracycline, Ann. Neurol., 51, 215-223, 2005.
Roberts M. C. Tetracycline Therapy: Update, Clin. Infect. Dis., 36, 462-467, 2003.
Sapadin A N and Fleischmajer R, Tetracyclines: Nonantibiotic Properties and their Clinical Implications, J. Am. Acad. Dermatol., 54, 258-265, 2006.
Skinner M. at al., Tetracycline in the Treatment of Rheumatoid Arthritis, Arthritis Rheum. 14, 727-732,1971.
Sompolinsky D. and Samra Z., Influence of Magnesium and Manganese on Some Biological and Physical Properties of Tetracycline, J. Bacteriol. 110, 468-476, 1972.
St. Clair E. W. et al., The Effects of Intravenous Doxycycline Therapy for Rheumatoid Arthritis, Arthritis. Rheum, 44, 1043-1047, 2001.
Stone M. et al, Should Tetracycline Treatment Be Used More Extensively for Rheumatoid Arthritis? Metaanalysis Demonstrate Clinical Benefit with Reduction in Disease Activity, J. Rheumatol, 30, 2112-2022, 2003.
Toussirot E. A., Oral Tolerance in the Treatment of Rheumatoid Arthritis, Drug Inflamm. Allergy, 1, 45-52, 2002.
Vibramycin, http://dailymed.nlm.nih. gov
Webster G. F. et al., Suppression of Polymorphonuclear Leukocyte Chemotactic Factor production in Propionobacterium acnes by Subminimal Inhibitory Concentrations of Tetracycline, Ampicilin, Minocycline, and Erythromycin, Antimicrob. Agents Chemother. 21, 770-772, 1982.
Weinberg M. A., New Applications of Doxycycline Hyclate in Medicine and Dentistry, US Pharmacist, 29, 04, 2004, http://uspharmacist.com.
Welling P. G., Koch P. A, Curtis C. L and Craig W. A., Bioavailability of Tetracycline and Doxycycline in fasted and Nonfasted Subjects, Antimicrob. Agents Chemother., 11, 462-469, 1976.
Wellen K. E. and Hotamisigil G. Inflammation, Stress, and Diabetes, J. Clin. Invest. 115, 1111-1119, 2005.
Worbs T et al, Oral Tolerance Originates in the Intestinal Immune System and Relies on the Antigen Carriage by Dendritic Cells, J. Exp. Med. 203, 519-527, 2006.
Wyeth Pharmaceuticals, UK, Minocin MR, SPC; 2005, http://emc.medicines.org.uk

What is claimed is:

1. A pharmaceutical composition for oral administration comprising one or more tetracycline compounds and one or more effective inhibitors of tetracycline compound absorption.

2. A composition of claim 1 comprising a tetracycline compound selected from natural tetracyclines, semi-synthetic tetracyclines, synthetic tetracyclines, and combinations thereof.

3. A composition of claim 2 comprising from about 0.5 mmole to about 4 mmoles of the tetracycline compound per dose.

4. A composition of claim 2 comprising from about 0.2 mmole to 0.5 mmole of the tetracycline compound per dose.

5. A composition of claim 2 comprising from about 0.02 mmole to 0.2 mmole of the a tetracycline compound per dose.

6. A composition of claim 1 wherein tetracycline absorption inhibitor comprises one or more non-toxic polyvalent metals, wherein said polyvalent metals are in form of a salt or a compound that can be converted in stomach or intestine to a salt.

7. A composition of claim 6 wherein the molar ratio of polyvalent metal(s) is higher than about 10 moles and lower than about 75 moles of said metal per one mole of tetracycline compound.

8. A composition of claim 6 wherein the molar ratio of polyvalent metal(s) is in the range from about 15 moles to about 40 moles per one mole of tetracycline compound.

9. A composition of claim 6 wherein the polyvalent metals comprise calcium, magnesium and combinations thereof.

10. A composition of claim 9 further comprising a non-toxic amount of iron, zinc, and combinations thereof.

11. A composition of claim 1 further comprising pharmaceutically active adjunct components.

12. A composition of claim 11 wherein the active adjunct components is selected from antibiotics, steroid anti-inflammatory compound(s), non-steroid anti-inflammatory compound, hormones, health supplements, vitamins, microelements, menthol, and combinations thereof.

13. A composition of claim 1 further comprising excipients selected from carriers, binders, lubricants, disintegrants, tableting agents, and combinations thereof.

14. A composition of claim 1 in the form of a solid, paste, powder, gel, suspension or solution.

15. A composition of claim 1 formulated as a gastric composition.

16. A composition of claim 1 formulated as an enteric composition.

17. A composition of claim 16 wherein release in the intestine of the tetracycline compounds and polyvalent metals is pH-dependent and is activated at pH ranging from about 5 to about 7.

18. A method for treating inflammatory disorders in a peripheral site comprising administering a non-protein anti-inflammatory compound such that it exerts its anti-inflammatory activity in the small intestine.

19. A method of claim 18 wherein the inflammatory disorder is induced or facilitated by food- or bacteria-derived compounds present in the intestine.

20. A method for treating inflammatory disorders in a peripheral site(s) comprising administering components comprising a tetracycline compound and an inhibitor of gastric absorption of said tetracycline compound, to facilitate the Tetracycline compound's anti-inflammatory activity in the intestine.

21. A method of claim 20 wherein the components are part of an orally administered gastric or an orally administered enteric composition.

22. A method according to claim 20 wherein the tetracycline compound is part of an oral composition comprising an inhibitor of gastric absorption of the tetracycline compound.

23. A method according to claim 20 wherein the tetracycline compound is selected from natural tetracyclines, semi-synthetic tetracyclines, synthetic tetracyclines, and combinations thereof.

24. A method according to claim 23 wherein the tetracycline compound is selected from tetracycline, oxytetracycline, chlortetracycline, doxycycline, pharmaceutically-acceptable salts thereof, and mixtures thereof.

25. A method of claim 20 wherein the tetracycline absorption inhibitor comprises one or more non-toxic polyvalent metals in the form of a salt or a compound that can be converted in the stomach or intestine to a salt.

26. A method of claim 20 wherein the components are administered together with a meal.

27. A method of claim 20 wherein the components are not administered together with a meal.

28. A method of claim 20 wherein the disorders treated are selected from diabetes, obsesity, atherosclerosis, cataracts, reperfusion injury, cancer, postinfections meningitis, rheumatic fever, systemic lupus erythenatosus, rheumatoid arthritis, acne, rosacea, autoimmune encephalitis, oveitis, thyroiditis, myasthenia, asthma, allergy, colitis, stroke, epilepsy, brain trauma, multiple sclerosis, Parkinson's disease, Alzheimer's disease, irritable bowel syndrome and Crohn's disease.
